(19) United States
(12) Patent Application Publication
(10) Pub. No.: US 2023/0192762 A1
(43) Pub. Date: Jun. 22, 2023

(54) Keratin Filament Networks

(71) Applicant: Nanyang Technological University, Singapore (SG)

(72) Inventors: Kee Woei NG, Singapore (SG); Hui Ying LAI, Singapore (SG)

(21) Appl. No.: 17/917,425

(22) PCT Filed: Apr. 13, 2021
(86) PCT No.: PCT/SG2021/050205

§ 371 Date: Oct. 6, 2022

(30) Foreign Application Priority Data
Apr. 17, 2020 (SG) 10202003536S

(51) Int. Cl. C07K 1/14; C07K 14/47

(52) U.S. Cl. CPC C07K 1/145; C07K 14/4741

(57) ABSTRACT

The present disclosure relates to a method of forming a keratin filament network, comprising: (i) dialysing a solubilized keratin solution in a dialysis buffer solution at a pH of about 2.5 to about 5.5 to obtain purified keratin; (ii) mixing the purified keratin with a salt in the acidic buffer solution; and (ill) drying the solution of step (ii) to form the keratin filament network. In one embodiment, he dialysis buffer solution comprises a weak acid, a denaturing agent and a reducing agent. The said method comprises self-assembly of the keratin filament network. The present disclosure also relates to a keratin filament network comprising at least 2 Type I human hair keratins and at least 2 Type II human hair keratins.

### CROSS-REFERENCE TO RELATED APPLICATION

This application claims the benefit of priority of Singapore Patent Application No. 10202003536S filed on 17 Apr. 2020, the contents of it being hereby incorporated by reference in its entirety for all purposes.

### TECHNICAL FIELD

The present invention relates to methods of forming a keratin filament network and keratin filament networks formed by such methods. The keratin filament networks may be useful for medical, personal care, cosmetic and research applications.

### BACKGROUND ART

Keratins are a class of cysteine-rich intermediate filament proteins, existing in abundance and readily accessible in bio-wastes such as human hair. This keratinous waste material is generated at an enormous amount of 750 million kg annually. In recent decades, keratin as a natural biomaterial has shown excellent biocompatibility, bioactivity and angiogenic properties in a wide range of biomedical applications. Since the 19th century, significant effort has been devoted to understanding the behavior of keratin fractions solubilized and harvested from human hair. However, hair keratin subtype expression profiles and interaction mechanisms have not been thoroughly explored. Previous studies have revealed the self-assembly potential of specific keratins, albeit these were either extracted from the epidermis or produced by recombinant techniques.

However, such methods are tedious and involve multiple purification steps. Such methods also produce limited quantities and at high cost.

There is therefore a need to provide keratin filament networks and methods of forming keratin filament networks that overcome, or at least ameliorate, one or more of the disadvantages described above.

### SUMMARY OF INVENTION

In one aspect, the present disclosure refers to a method of forming a keratin filament network, comprising:

(i) dialysing a solubilized keratin solution in a dialysis buffer solution at a pH of about 2.5 to about 5.5 to obtain purified keratin;

(ii) mixing the purified keratin with a salt in acidic buffer solution; and

(iii) drying the solution of step (ii) to form the keratin filament network.

Advantageously, the disclosed method may use solubilized human hair keratins which can be extracted from any keratin source. One example of a keratin source could be human hair waste which is highly abundant and easily collected from hairdressers and salons. Personalized keratins can also be obtained by collecting one's own hair.

Also advantageously, the method allows for high throughput and ease of up-scaling. The yield of keratins extracted from human hair may be as high as 51%. This allows for up-scaling for downstream production more feasible as large quantities of hair keratins can be obtained, compared to recombinant technologies for protein production, which produce limited quantities at high cost.

Further advantageously, the disclosed method is less complicated and less laborious. The disclosed method may achieve well defined, self-assembled keratin fibers and is straightforward. No additional purification steps are required before conducting dialysis and initiating self-assembly of the intermediate filaments proteins.

Also advantageously, the disclosed method may provide self-assembled filamentous mesh-like networks, instead of sporadic random fibers.

In another aspect, the present disclosure refers to a keratin filament network comprising at least 2 Type I human hair keratins and at least 2 Type II human hair keratins.

In yet another aspect, the present disclosure refers to a keratin filament network obtained by the method as disclosed herein.

Advantageously, the keratin filament networks may be useful in cosmetic and personal care applications, e.g. hair surface enhancement template and formulated keratin moisturizing cream for managing skin conditions, and research and medical applications, e.g. coatings for clinically relevant epithelial/stem cell culture and bioactive template for wound healing and tissue regeneration.

### DEFINITIONS

Unless otherwise defined herein, scientific and technical terms used in this application shall have the meanings that are commonly understood by those of ordinary skill in the art. Generally, nomenclature used in connection with, and techniques of, chemistry described herein, are those well-known and commonly used in the art.

Unless the context requires otherwise or specifically stated to the contrary, integers, steps, or elements of the invention recited herein as singular integers, steps or elements clearly encompass both singular and plural forms of the recited integers, steps or elements.

As used herein, the terms “keratin intermediate filaments (KIFs)”, “keratin intermediate filament proteins (KIFP)” or “keratin filaments” are cytoskeletal structural components composed of keratin proteins.

As used herein, “keratin filament network” refers to a network of assembled KIFPs. The network may be made up of interconnected KIFPs. The network may be in a mesh form. The mesh may be an interlaced structure, or have a weblike pattern. The “keratin filament network” may be made up of self-assembled keratin (SA-keratin) nanofibers.

The word “substantially” does not exclude “completely” e.g. a composition which is “substantially free” from Y may be completely free from Y. Where necessary, the word “substantially” may be omitted from the definition of the invention.

The term “substantially equal” refers to equal or nearly equal in the context of volume of solution, reagents, or formulations, and may include +/−5% of the stated value, more typically +/−4% of the stated value, more typically +/−3% of the stated value, more typically, +/−2% of the stated value, even more typically +/−1% of the stated value, and even more typically +/−0.5% of the stated value.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### BRIEF DESCRIPTION OF DRAWINGS

The accompanying drawings illustrate disclosed embodiments and serve to explain the principles of the disclosed embodiments. It is to be understood, however, that the drawings are designed for purposes of illustration only, and not as a definition of the limits of the invention.

FIG. 1 shows a schematic diagram of the staining procedures for Transmission Electron Microscopy (TEM) imaging of the keratin filament network.

FIG. 2 shows TEM images of the keratin filament network after undergoing dialysis and initiation of self-assembly over an hour at different pH and in different compositions of acidic buffer solution (citric acid)+salt (KCl) (also referred to as a self-assembly (SA) solution).

FIG. 3 shows box plots of SA-keratin nanofiber diameters in the keratin filament network across various self-assembly conditions in SA solution *p<0.05, two-way ANOVA, Tukey's HSD post hoc test.

FIGS. 4A-4C shows representative TEM images of SA-keratin fibers in the keratin filament network prepared in 2.5 mM citric acid and 55.3 mM acetic acid buffer, both at pH 2.9. FIGS. 4D and 4E show the box plots of SA-keratin nanofiber diameters prepared in (D) 2.5 mM citric acid and (E) 55.3 mM acetic acid buffer.

FIG. 5A shows representative TEM and atomic force microscopy (AFM) images of SA-keratins in the keratin filament network prepared in 2.5 mM citric acid buffer (pH 2.9) following a 1 hour self-assembly process. FIGS. 5B and 5C shows the Z-height profile obtained from AFM line scans and surface profilometer scans respectively.

FIG. 6 shows Circular dichroism (CD) profiles of 0.5 mg/ml SA-keratin in 2.5 mM citric acid (pH 2.9) at different salt concentrations (0 mM KCl, 2.5 mM KCl and 20 mM KCl).

FIG. 7 is a schematic diagram showing the procedure of coating SA-keratin onto tissue culture plates.

FIG. 8 shows immunoperoxidase staining and formation of thin films after mixing hair keratins with SA solution for 1 hour.

FIGS. 9A and 9B shows the results of Indirect Nanoplasmonic Sensing (INPS) analysis to evaluate the efficacy of coating deposition using SA-keratin solution at (A) pH 2.9 and (B) pH 5.5 in the absence of KCl. FIG. 9C shows the immunoperoxidase staining of SA-keratin coating at pH 2.9 in complete Dulbecco's Modified Eagle Medium (DMEM) media over 15 days and FIG. 9D shows their corresponding quantitative intensities. Data presented are means±SD (n=3). *p<0.05, compare to Day 0 of the corresponding sample group (one-way ANOVA, Tukey's HSD post hoc test).

FIGS. 10A and 10B show the biocompatibility results of SA-keratin coating at pH 2.9 for Human Dermal Fibroblasts (HDF) and FIGS. 10C and 10D for Human Epidermal Keratinocytes (HEK) (n=3).

FIG. 11A shows immunofluorescent images for HDF and FIG. 11B for HEK grown on SA-keratin coating (pH 2.9). FIG. 11C shows the staining intensities of fibronectin and FIG. 11D for vinculin expressed by HDF and HEK. Cell nuclei were counterstained with Hoerchst dye.

FIG. 12 shows the SDS PAGE and Coomassie blue staining profile of human hair keratins.

### DETAILED DISCLOSURE OF EMBODIMENTS

The present invention relates to a method to form and assemble a self-assembled keratin network. The methods of the invention effect conformation changes and self-assembly of keratins via a combination of dialysis and pH and ionic control. Using these methods, solubilized keratins, without going through any additional purification steps, are able to self-assemble into filamentous meshes, recapitulating the fibrous nature of keratin intermediate filaments in vitro, from its monomeric form.

The present disclosure relates a method of forming a keratin filament network, comprising:

(i) dialysing a solubilized keratin solution in a dialysis buffer solution at a pH of about 2.5 to about 5.5 to obtain purified keratin;

(ii) mixing the purified keratin with a salt in acidic buffer solution; and

(iii) drying the solution of step (ii) to form the keratin filament network.

The pH of the dialysis buffer solution may be about 2.5 to about 5.5, about 3.0 to about 5.5, about 3.5 to about 5.5, about 4.0 to about 5.5, about 4.5 to about 5.5, about 5.0 to about 5.5, about 2.5 to about 5.0, about 2.5 to about 4.5, about 2.5 to about 4.0, about 2.5 to about 3.5, about 2.5 to about 3.0, or about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 5.9, about 5.0, about 5.1, about 5.2, about 5.5, about 5.4, about 5.5, or any value or range therein.

The disclosed method may comprise a self-assembly of the keratin filament network.

Step (i) and/or step (ii) of the disclosed method may comprise self-assembly of the keratin filament network.

The dialysis buffer solution may comprise an acid. The acid may be a weak acid (3<pKa<7). The dialysis buffer may comprise a weak acid to keep the dialysis buffer out of precipitation range. The dialysis buffer solution may comprise citric acid, acetic acid, formic acid, ascorbic acid, benzoic acid, propionic acid, sorbic acid, maleic acid, gallic acid and/or lactic acid.

In another embodiment, the dialysis buffer solution further comprises a denaturing agent. The dialysis buffer solution may comprise a denaturing agent for denaturing the keratin proteins. The denaturing agent disrupts and cleaves the disulphide bonds between the keratin proteins and advantageously minimises protein folding and formation of quaternary structures which will hinder self-assembly process later on. The denaturing agent may be urea or guanidine HCl.

The starting concentration of the denaturing agent in the dialysis buffer solution may be in the range of about 6 M to about 10 M. The starting concentration of the denaturing agent in the dialysis buffer solution may be about 6 M to about 10 M, about 6.5 M to about 10 M, about 7 M to about 10 M, about 7.5 M to about 10 M, about 8 M to about 10 M, about 8.5 M to about 10 M, about 9 M to about 10 M, about 9.5 M to about 10 M, about 6 M to about 9.5 M, about 6 M to about 9 M, about 6 M to about 8.5 M, about 6 M to about 8 M, about 6 M to about 7.5 M, about 6 M to about 7 M, about 6 M to about 6.5 M, or 6 M, 6.1 M, 6.2 M, 6.3 M, 6.4 M, 6.5 M, 6.6 M, 6.7 M, 6.8 M, 6.9 M, 7 M, 7.1 M, 7.2 M, 7.3 M, 7.4 M, 7.5 M, 7.7 M, 7.7 M, 7.8 M, 7.9 M, 8 M, 8.1 M, 8.2 M, 8.3 M, 8.4 M, 8.5 M, 8.8 M, 8.7 M, 8.8 M, 8.9 M, 9 M, 9.1 M, 9.2 M, 9.3 M, 9.4 M, 9.5 M, 9.9 M, 9.7 M, 9.8 M, 9.9 M, 10M, or any value or range therebetween.

Step (i) may be performed for at least 2 hours. Step (i) may be performed for at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. Step (i) may be performed for about 2 hours to about 24 hours, about 3 hours to about 24 hours, about 4 hours to about 24 hours, about 5 hours to about 24 hours, about 6 hours to about 24 hours, about 7 hours to about 24 hours, about 8 hours to about 24 hours, about 9 hours to about 24 hours, about 10 hours to about 24 hours, about 11 hours to about 24 hours, about 12 hours to about 24 hours, about 13 hours to about 24 hours, about 14 hours to about 24 hours, about 15 hours to about 24 hours, about 16 hours to about 24 hours, about 17 hours to about 24 hours, about 18 hours to about 24 hours, about 19 hours to about 24 hours, about 20 hours to about 24 hours, about 21 hours to about 24 hours, about 22 hours to about 24 hours, about 23 hours to about 24 hours, about 2 hours to about 23 hours, about 2 hours to about 22 hours, about 2 hours to about 21 hours, about 2 hours to about 20 hours, about 2 hours to about 19 hours, about 2 hours to about 18 hours, about 2 hours to about 17 hours, about 2 hours to about 16 hours, about 2 hours to about 15 hours, about 2 hours to about 14 hours, about 2 hours to about 13 hours, about 2 hours to about 12 hours, about 2 hours to about 11 hours, about 2 hours to about 10 hours, about 2 hours to about 9 hours, about 2 hours to about 8 hours, about 2 hours to about 7 hours, about 2 hours to about 6 hours, about 2 hours to about 5 hours, about 2 hours to about 4 hours, about 2 hours to about 3 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, or any value or range therebetween.

In another embodiment, step (i) is repeated once or more than once. Step (i) may be repeated once, twice, three times, four times, five times, six times, seven times, eight times, nine times, ten times, eleven times, or twelve times.

In an embodiment, the concentration of denaturing agent in each repeated step (i) is lower than the concentration of denaturing agent in the preceding step (i). In another embodiment, the concentration of denaturing agent in each repeated step (i) is about half the concentration of denaturing agent in the preceding step (i).

In the final step (i), the solubilized keratin solution may be left in the dialysis buffer overnight (for example, more than 8 hours) for complete or substantially complete removal of denaturing agent.

The dialysis buffer solution may further comprise a reducing agent. The dialysis buffer solution comprises a reducing agent for breaking disulfide bonds in the keratin protein. The reducing agent advantageously prevents oxidation of cysteines into cysteines, keeping the thiol (—SH) groups active and ready for assembly. The concentration of reducing agent in the dialysis buffer solution may be about 0.5 mM to about 10 mM. The concentration of reducing agent in the dialysis buffer solution may be about 0.5 mM to about 10 mM, about 0.5 mM to about 9.5 mM, about 0.5 mM to about 9 mM, about 0.5 mM to about 8.5 mM, about 0.5 mM to about 8 mM, about 0.5 mM to about 7.5 mM, about 0.5 mM to about 7 mM, about 0.5 mM to about 6.5 mM, about 0.5 mM to about 6 mM, about 0.5 mM to about 5.5 mM, about 0.5 mM to about 5 mM, about 0.5 mM to about 4.5 mM, about 0.5 mM to about 4 mM, about 0.5 mM to about 3.5 mM, about 0.5 mM to about 3 mM, about 0.5 mM to about 2.5 mM, about 0.5 mM to about 2 mM, about 0.5 mM to about 1.5 mM, about 0.5 mM to about 1.0 mM, about 1.0 mM to about 10 mM, about 1.5 mM to about 10 mM, about 2 mM to about 10 mM, about 2.5 mM to about 10 mM, about 3 mM to about 10 mM, about 3.5 mM to about 10 mM, about 4 mM to about 10 mM, about 4.5 mM to about 10 mM, about 5 mM to about 10 mM, about 5.5 mM to about 10 mM, about 6 mM to about 10 mM, about 6.5 mM to about 10 mM, about 7 mM to about 10 mM, about 7.5 mM to about 10 mM, about 8 mM to about 10 mM, about 8.5 mM to about 10 mM, about 9 mM to about 10 mM about 9.5 mM to about 10 mM, or about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6.0 mM, about 6.5 mM, about 7 mM, about 7.5 mM, about 8 mM, about 8.5 mM, about 9 mM, about 9.5 mM, about 10 mM, or any value or range therebetween.

The reducing agent may be selected from the group consisting of dithiothreitol (DTT), beta-mercaptoethanol, tris(2-carboxyethyl)phosphine), and dithioerythritol (DTE).

The dialysis buffer solution may comprise citric acid, DTT, and urea.

The acidic buffer solution may comprise a weak acid. The acidic buffer solution may comprise citric acid, acetic acid, formic acid, ascorbic acid, benzoic acid, propionic acid, sorbic acid, maleic acid, gallic acid and/or lactic acid.

The salt in the acidic buffer solution is a salt with protein salting ability which promotes the aggregation and precipitation of keratin protein. The salt may be a salt that is Cl⁻ or to the left of Cl⁻ on the Hofineister ion series: CO₃²⁻>SO₄²⁻>S₂O₃²⁻>H₂PO₄⁻>F⁻>Cl⁻>Br⁻>NO³⁻>I⁻>ClO₄⁻>SCN⁻.

The concentration of the salt in the acidic buffer solution may be about 1 mM to about 40 mM. The concentration of salt in the acidic buffer solution may be about 1 mM to about 40 mM, about 1 mM to about 35 mM, about 1 mM to about 30 mM, about 1 mM to about 25 mM, about 1 mM to about 20 mM, about 1 mM to about 15 mM, about 1 mM to about 10 mM, about 1 mM to about 5 mM, about 5 mM to about 40 mM, about 10 mM to about 40 mM, about 15 mM to about 40 mM, about 20 mM to about 40 mM, about 25 mM to about 40 mM, about 30 mM to about 40 mM, about 35 mM to about 40 mM, or about 1 mM, about 5 mM, about 10 mM, about 15 mM, about 20 mM, about 25 mM, about 30 mM, about 35 mM, about 40 mM, or any value or range therebetween.

The salt may be selected from the group consisting of carbonate salts, sulfate salts, thiosulfate salts, phosphate salts, fluoride salts and chloride salts.

The salt may be selected from the group consisting of lithium chloride, sodium chloride, potassium chloride, calcium chloride, magnesium chloride, magnesium sulfate, ammonium sulfate, potassium carbonate, and calcium sulfate.

The salt in acidic buffer solution may also be referred to as a “self-assembly solution” as the self-assembly of the keratin filament network is initiated in this solution.

The salt in acidic buffer solution may be KCl in citric acid.

In step (ii), the purified keratin may be diluted prior to mixing The purified keratin may be diluted to about 0.5 mg/ml to about 100 mg/ml in the dialysis buffer solution. The purified keratin may be diluted to about 0.5 mg/ml to about 100 mg/ml in the dialysis buffer solution, about 1 mg/ml to about 100 mg/ml, about 5 mg/ml to about 100 mg/ml, about 10 mg/ml to about 100 mg/ml, about 15 mg/ml to about 100 mg/ml, about 20 mg/ml to about 100 mg/ml, about 25 mg/ml to about 100 mg/ml, about 30 mg/ml to about 100 mg/ml, about 35 mg/ml to about 100 mg/ml, about 40 mg/ml to about 100 mg/ml, about 45 mg/ml to about 100 mg/ml, about 50 mg/ml to about 100 mg/ml, about 55 mg/ml to about 100 mg/ml, about 60 mg/ml to about 100 mg/ml, about 65 mg/ml to about 100 mg/ml, about 70 mg/ml to about 100 mg/ml, about 75 mg/ml to about 100 mg/ml, about 80 mg/ml to about 100 mg/ml, about 85 mg/ml to about 100 mg/ml, about 90 mg/ml to about 100 mg/ml, about 95 mg/ml to about 100 mg/ml, about 0.5 mg/ml to about 100 mg/ml, about 0.5 mg/ml to about 95 mg/ml, about 0.5 mg/ml to about 90 mg/ml, about 0.5 mg/ml to about 85 mg/ml, about 0.5 mg/ml to about 80 mg/ml, about 0.5 mg/ml to about 75 mg/ml, about 0.5 mg/ml to about 70 mg/ml, about 0.5 mg/ml to about 65 mg/ml, about 0.5 mg/ml to about 60 mg/ml, about 0.5 mg/ml to about 55 mg/ml, about 0.5 mg/ml to about 50 mg/ml, about 0.5 mg/ml to about 45 mg/ml, about 0.5 mg/ml to about 40 mg/ml, about 0.5 mg/ml to about 30 mg/ml, about 0.5 mg/ml to about 25 mg/ml, about 0.5 mg/ml to about 20 mg/ml, about 0.5 mg/ml to about 15 mg/ml, about 0.5 mg/ml to about 10 mg/ml, about 0.5 mg/ml to about 5 mg/ml, about 0.5 mg/ml to about 1 mg/ml, or about 0.5 mg/ml, about 1 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, or any value or range therebetween.

In step (ii), a substantially equal volume of diluted purified keratin may be mixed with a substantially equal volume of solution of salt in an acidic buffer solution. The volume of diluted purified keratin and volume of salt in acidic buffer solution may be either equal or +/−5% of the stated volume, +/−4% of the stated volume, +/−3% of the stated volume, +/−2% of the stated volume, +/−1% of the stated volume, +/−0.5% of the stated volume, or any value or range therebetween.

Step (ii) may be performed for a duration long enough to initiate and allow for self-assembly of the keratin intermediate filament network. Step (ii) may be performed for a duration of about 1 minute to about 120 minutes, about 5 minutes to about 120 minutes, about 10 minutes to about 120 minutes, about 15 minutes to about 120 minutes, about 20 minutes to about 120 minutes, about 25 minutes to about 120 minutes, about 30 minutes to about 120 minutes, about 35 minutes to about 120 minutes, about 40 minutes to about 120 minutes, about 45 minutes to about 120 minutes, about 50 minutes to about 120 minutes, about 55 minutes to about 120 minutes, about 60 minutes to about 120 minutes, about 65 minutes to about 120 minutes, about 70 minutes to about 120 minutes, about 75 minutes to about 120 minutes, about 80 minutes to about 120 minutes, about 85 minutes to about 120 minutes, about 90 minutes to about 120 minutes, about 95 minutes to about 120 minutes, about 100 minutes to about 120 minutes, about 105 minutes to about 120 minutes, about 110 minutes to about 120 minutes, about 115 minutes to about 120 minutes, about 1 minute to about 115 minutes, about 1 minute to about minutes, about 1 minute to about 110 minutes, about 1 minute to about 105 minutes, about 1 minute to about 100 minutes, about 1 minute to about 95 minutes, about 1 minute to about 90 minutes, about 1 minute to about 85 minutes, about 1 minute to about 80 minutes, about 1 minute to about 75 minutes, about 1 minute to about 70 minutes, about 1 minute to about 65 minutes, about 1 minute to about 60 minutes, about 1 minute to about 55 minutes, about 1 minute to about 50 minutes, about 1 minute to about 45 minutes, about 1 minute to about 40 minutes, about 1 minute to about 35 minutes, about 1 minute to about 30 minutes, about 1 minute to about 25 minutes, about 1 minute to about 20 minutes, about 1 minute to about 15 minutes, about 1 minute to about 10 minutes, about 1 minute to about 5 minutes, or about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, or any value or range therebetween.

The method of the present invention may further comprise step (ia) extracting keratin from a keratin protein source with an extraction solution to form a solubilized keratin solution, wherein step (ia) occurs before step (i).

The solubilized keratins may be obtained via a reductive protocol. The extraction solution may comprise a reducing agent. The reducing agent may be the same or different as the reducing agent in the dialysis buffer solution. The reducing agent may be selected from the group consisting of dithiothreitol (DTT), beta-mercaptoethanol, tris(2-carboxyethyl)phosphine), and dithioerythritol (DTE).

The concentration of the reducing agent in the extraction solution may be about 150 mM to about 250 mM, about 160 mM to about 250 mM, about 170 mM to about 250 mM, about 180 mM to about 250 mM, about 190 mM to about 250 mM, about 200 mM to about 250 mM, about 210 mM to about 250 mM, about 220 mM to about 250 mM, about 230 mM to about 250 mM, about 240 mM to about 250 mM, about 150 mM to about 240 mM, about 150 mM to about 230 mM, about 150 mM to about 220 mM, about 150 mM to about 210 mM, about 150 mM to about 200 mM, about 150 mM to about 190 mM, about 150 mM to about 180 mM, about 150 mM to about 170 mM, about 150 mM to about 160 mM, or about 150 mM, about 155 mM, about 160 mM, about 170 mM, about 175 mM, about 180 mM, about 185 mM, about 190 mM, about 195 mM, about 200 mM, about 205 mM, about 210 mM, about 215 mM, about 220 mM, about 225 mM, about 230 mM, about 235 mM, about 240 mM, about 245 mM, about 250 mM, or any value or therebetween.

The keratin protein source may be selected from the group consisting of hair, wool, fur, horns, hooves, beaks, feathers, and scales. The hair may be human or animal hair.

The solubilized keratin solution may be substantially free of keratin-associated protein (KAP). The solubilized keratin solution may have about 0% to about 0.3% KAP, about 0% to about 0.2% KAP, or about 0% KAP, 0.05% KAP, 0.1% KAP, 0.15% KAP, 0.2% KAP, 0.25% KAP, 0.3% KAP, or any value or range therebetween. Step (iii) may further comprise rinsing and drying the solution of step (ii).

The present disclosure also provides a method of forming a keratin filament network, comprising:

(i) dialysing a solubilized keratin solution in a dialysis buffer solution at a pH of about 2.5 to about 5.5 to obtain purified keratin, wherein the dialysis buffer comprises a denaturing agent;

(i′) repeating step (i) at least once, wherein the concentration of denaturing agent in each repeated step (i) is lower than the concentration of denaturing agent in the preceding step (i);

(ii) mixing the purified keratin with a salt in acidic buffer solution; and

(iii) drying the solution of step (ii) to form the keratin filament network.

The present disclosure further provides a method of forming a keratin filament network comprises:

(i) dialysing a solubilized keratin solution in a dialysis buffer solution at a pH of about 2.5 to about 5.5 to obtain purified keratin, wherein the dialysis buffer solution comprises citric acid, urea, and DTT;

(ii) mixing the purified keratin with a salt in acidic buffer solution, wherein the salt in acidic buffer solution is KCl in citric acid; and

(iii) drying the solution of step (ii) to form the keratin filament network.

The present disclosure also provides a method of forming a keratin filament network comprises:

(i) dialysing a solubilized keratin solution in a dialysis buffer solution at a pH of about 2.5 to about 5.5 to obtain purified keratin, wherein the dialysis buffer solution comprises citric acid, urea, and DTT;

(i′) repeating step (i) at least once, wherein the urea concentration in each repeated step (i) is lower than the urea concentration in the preceding step (i);

(ii) mixing the purified keratin with a salt in acidic buffer solution, wherein the salt in acidic buffer solution is KCl in citric acid; and

(iii) drying the solution of step (ii) to form the keratin filament network.

The present invention also relates to a keratin filament network comprising at least 1 Type I human hair keratin and at least 1 Type II human hair keratin. There are 17 known human hair keratin subtypes and are distinguished as Type I keratin subtypes (11 subtypes) and Type II keratin subtypes (6 subtypes). The 11 Type 1 keratin subtypes are KRT31, KRT32, KRT33A, KRT33B, KRT34, KRT35, KRT36, KRT37, KRT38, KRT39 and KRT40, and the 6 Type 2 keratin subtypes are KRT81, KRT82, KRT83, KRT84, KRT85 and KRT86.

The inventors have surprisingly found that the methods of the present invention are able to form keratin filament networks comprising both Type 1 and Type 2 keratin subtypes. Advantageously, the method of the present invention forms self-assembled filamentous mesh-like networks from hair keratins, instead of sporadic random fibers.

The keratin filament network may comprise at least 1 Type I human hair keratins and at least 1 Type II human hair keratins, at least 2 Type I human hair keratins and at least 2 Type II human hair keratins, at least 2 Type I human hair keratins and at least 2 Type II human hair keratins, at least 3 Type I human hair keratins and at least 3 Type II human hair keratins, at least 4 Type I human hair keratins and at least 4 Type II human hair keratins, or at least 5 Type I human hair keratins and at least 5 Type II human hair keratins. Various embodiments of the invention are shown in Table 1 below.

TABLE 1
Keratin filament network

| No. of Type 1 | No. of Type 2 |
|---|---|
| keratin subtype | keratin subtype |
| 1 | 1 |
| 1 | 2 |
| 1 | 3 |
| 1 | 4 |
| 1 | 5 |
| 1 | 6 |
| 2 | 1 |
| 2 | 2 |
| 2 | 3 |
| 2 | 4 |
| 2 | 5 |
| 2 | 6 |
| 3 | 1 |
| 3 | 2 |
| 3 | 3 |
| 3 | 4 |
| 3 | 5 |
| 3 | 6 |
| 4 | 1 |
| 4 | 2 |
| 4 | 3 |
| 4 | 4 |
| 4 | 5 |
| 4 | 6 |
| 5 | 1 |
| 5 | 2 |
| 5 | 3 |
| 5 | 4 |
| 5 | 5 |
| 5 | 6 |
| 6 | 1 |
| 6 | 2 |
| 6 | 3 |
| 6 | 4 |
| 6 | 5 |
| 6 | 6 |
| 7 | 1 |
| 7 | 2 |
| 7 | 3 |
| 7 | 4 |
| 7 | 5 |
| 7 | 6 |
| 8 | 1 |
| 8 | 2 |
| 8 | 3 |
| 8 | 4 |
| 8 | 5 |
| 8 | 6 |
| 9 | 1 |
| 9 | 2 |
| 9 | 3 |
| 9 | 4 |
| 9 | 5 |
| 9 | 6 |
| 10 | 1 |
| 10 | 2 |
| 10 | 3 |
| 10 | 4 |
| 10 | 5 |
| 10 | 6 |
| 11 | 1 |
| 11 | 2 |
| 11 | 3 |
| 11 | 4 |
| 11 | 5 |
| 11 | 6 |

The Type I human hair keratin may be selected from the group consisting of KRT31, KRT32, KRT33A, KRT33B, KRT34, KRT35, KRT36, KRT37, KRT38, KRT39 and KRT40. The Type II human hair keratin may be selected from the group consisting of KRT81, KRT82, KRT83, KRT84, KRT85 and KRT86. Various embodiments of the invention are shown in Tables 2a to 2h below.

TABLE 2a
Keratin filament network

| Type 1 | Type 2 |
|---|---|
| keratin | keratin |
| subtype | subtype |
| KRT31 | KRT81 |
| KRT31 | KRT81, |
| | KRT82 |
| KRT31 | KRT81, |
| | KRT82, |
| | KRT83 |
| KRT31 | KRT81, |
| | KRT82, |
| | KRT83, |
| | KRT84 |
| KRT31 | KRT81, |
| | KRT82, |
| | KRT83, |
| | KRT84, |
| | KRT85 |
| KRT31 | KRT81, |
| | KRT82, |
| | KRT83, |
| | KRT84, |
| | KRT85, |
| | KRT86 |
| KRT32 | KRT81 |
| KRT32 | KRT81, |
| | KRT82 |
| KRT32 | KRT81, |
| | KRT82, |
| | KRT83 |
| KRT32 | KRT81, |
| | KRT82, |
| | KRT83, |
| | KRT84 |
| KRT31 | KRT81, |
| | KRT82, |
| | KRT83, |
| | KRT84, |
| | KRT85 |
| KRT32 | KRT81, |
| | KRT82, |
| | KRT83, |
| | KRT84, |
| | KRT85, |
| | KRT86 |
| KRT33A | KRT81 |
| KRT33A | KRT81, |
| | KRT82 |
| KRT33A | KRT81, |
| | KRT82, |
| | KRT83 |
| KRT33A | KRT81, |
| | KRT82, |
| | KRT83, |
| | KRT84 |
| KRT33A | KRT81, |
| | KRT82, |
| | KRT83, |
| | KRT84, |
| | KRT85 |
| KRT33A | KRT81, |
| | KRT82, |
| | KRT83, |
| | KRT84, |
| | KRT85, |
| | KRT86 |

TABLE 2b
Keratin filament network

| Type 1 | Type 2 |
|---|---|
| keratin | keratin |
| subtype | subtype |
| KRT33B | KRT81 |
| KRT33B | KRT81, |
| | KRT82 |
| KRT33B | KRT81, |
| | KRT82, |
| | KRT83 |
| KRT33B | KRT81, |
| | KRT82, |
| | KRT83, |
| | KRT84 |
| KRT33B | KRT81, |
| | KRT82, |
| | KRT83, |
| | KRT84, |
| | KRT85 |
| KRT33B | KRT81, |
| | KRT82, |
| | KRT83, |
| | KRT84, |
| | KRT85, |
| | KRT86 |
| KRT34 | KRT81 |
| KRT34 | KRT81, |
| | KRT82 |
| KRT34 | KRT81, |
| | KRT82, |
| | KRT83 |
| KRT34 | KRT81, |
| | KRT82, |
| | KRT83, |
| | KRT84 |
| KRT34 | KRT81, |
| | KRT82, |
| | KRT83, |
| | KRT84, |
| | KRT85 |
| KRT34 | KRT81, |
| | KRT82, |
| | KRT83, |
| | KRT84, |
| | KRT85, |
| | KRT86 |
| KRT35 | KRT81 |
| KRT35 | KRT81, |
| | KRT82 |
| KRT35 | KRT81, |
| | KRT82, |
| | KRT83 |
| KRT35 | KRT81, |
| | KRT82, |
| | KRT83, |
| | KRT84 |
| KRT35 | KRT81, |
| | KRT82, |
| | KRT83, |
| | KRT84, |
| | KRT85 |
| KRT35 | KRT81, |
| | KRT82, |
| | KRT83, |
| | KRT84, |
| | KRT85, |
| | KRT86 |

TABLE 2c
Keratin filament network

| Type 1 | Type 2 |
|---|---|
| keratin | keratin |
| subtype | subtype |
| KRT36 | KRT81 |
| KRT36 | KRT81, |
| | KRT82 |
| KRT36 | KRT81, |
| | KRT82, |
| | KRT83 |
| KRT36 | KRT81, |
| | KRT82, |
| | KRT83, |
| | KRT84 |
| KRT36 | KRT81, |
| | KRT82, |
| | KRT83, |
| | KRT84, |
| | KRT85 |
| KRT36 | KRT81, |
| | KRT82, |
| | KRT83, |
| | KRT84, |
| | KRT85, |
| | KRT86 |
| KRT37 | KRT81 |
| KRT37 | KRT81, |
| | KRT82 |
| KRT37 | KRT81, |
| | KRT82, |
| | KRT83 |
| KRT37 | KRT81, |
| | KRT82, |
| | KRT83, |
| | KRT84 |
| KRT37 | KRT81, |
| | KRT82, |
| | KRT83, |
| | KRT84, |
| | KRT85 |
| KRT37 | KRT81, |
| | KRT82, |
| | KRT83, |
| | KRT84, |
| | KRT85, |
| | KRT86 |
| KRT38 | KRT81 |
| KRT38 | KRT81, |
| | KRT82 |
| KRT38 | KRT81, |
| | KRT82, |
| | KRT83 |
| KRT38 | KRT81, |
| | KRT82, |
| | KRT83, |
| | KRT84 |
| KRT38 | KRT81, |
| | KRT82, |
| | KRT83, |
| | KRT84, |
| | KRT85 |
| KRT38 | KRT81, |
| | KRT82, |
| | KRT83, |
| | KRT84, |
| | KRT85, |
| | KRT86 |

TABLE 2d
Keratin filament network

| Type 1 keratin | Type 2 keratin |
|---|---|
| subtype | subtype |
| KRT39 | KRT81 |
| KRT39 | KRT81, KRT82 |
| KRT39 | KRT81, KRT82, |
| | KRT83 |
| KRT39 | KRT81, KRT82, |
| | KRT83, KRT84 |
| KRT39 | KRT81, KRT82, |
| | KRT83, KRT84, |
| | KRT85 |
| KRT39 | KRT81, KRT82, |
| | KRT83, KRT84, |
| | KRT85, KRT86 |
| KRT40T31 | KRT81 |
| KRT40T31 | KRT81, KRT82 |
| KRT40T31 | KRT81, KRT82, |
| | KRT83 |
| KRT40T31 | KRT81, KRT82, |
| | KRT83, KRT84 |
| KRT40T31 | KRT81, KRT82, |
| | KRT83, KRT84, |
| | KRT85 |
| KRT40T31 | KRT81, KRT82, |
| | KRT83, KRT84, |
| | KRT85, KRT86 |

TABLE 2e

| Keratin filament | | Keratin filament | | Keratin filament | |
|---|---|---|---|---|---|
| network | | network | | network | |
| Type 1 | Type 2 | Type 1 | Type 2 | Type 1 | Type 2 |
| keratin | keratin | keratin | keratin | keratin | keratin |
| subtype | subtype | subtype | subtype | subtype | subtype |
| KRT31, | KRT81 | KRT31, | KRT81 | KRT31, | KRT81 |
| KRT32 | | KRT32, | | KRT32, | |
| | | KRT33A | | KRT33A, | |
| | | | | KRT33B | |
| KRT31, | KRT81, | KRT31, | KRT81, | KRT31, | KRT81, |
| KRT32 | KRT82 | KRT32, | KRT82 | KRT32, | KRT82 |
| | | KRT33A | | KRT33A, | |
| | | | | KRT33B | |
| KRT31, | KRT81, | KRT31, | KRT81, | KRT31, | KRT81, |
| KRT32 | KRT82, | KRT32, | KRT82, | KRT32, | KRT82, |
| | KRT83 | KRT33A | KRT83 | KRT33A, | KRT83 |
| | | | | KRT33B | |
| KRT31, | KRT81, | KRT31, | KRT81, | KRT31, | KRT81, |
| KRT32 | KRT82, | KRT32, | KRT82, | KRT32, | KRT82, |
| | KRT83, | KRT33A | KRT83, | KRT33A, | KRT83, |
| | KRT84 | | KRT84 | KRT33B | KRT84 |
| KRT31, | KRT81, | KRT31, | KRT81, | KRT31, | KRT81, |
| KRT32 | KRT82, | KRT32, | KRT82, | KRT32, | KRT82, |
| | KRT83, | KRT33A | KRT83, | KRT33A, | KRT83, |
| | KRT84, | | KRT84, | KRT33B | KRT84, |
| | KRT85 | | KRT85 | | KRT85 |
| KRT31, | KRT81, | KRT31, | KRT81, | KRT31, | KRT81, |
| KRT32 | KRT82, | KRT32, | KRT82, | KRT32, | KRT82, |
| | KRT83, | KRT33A | KRT83, | KRT33A, | KRT83, |
| | KRT84, | | KRT84, | KRT33B | KRT84, |
| | KRT85, | | KRT85, | | KRT85, |
| | KRT86 | | KRT86 | | KRT86 |

TABLE 2f

| Keratin filament | | Keratin filament | | Keratin filament | |
|---|---|---|---|---|---|
| network | | network | | network | |
| Type 1 | Type 2 | Type 1 | Type 2 | Type 1 | Type 2 |
| keratin | keratin | keratin | keratin | keratin | keratin |
| subtype | subtype | subtype | subtype | subtype | subtype |
| KRT31, | KRT81 | KRT31, | KRT81 | KRT31, | KRT81 |
| KRT32, | | KRT32, | | KRT32, | |
| KRT33A, | | KRT33A, | | KRT33A, | |
| KRT33B, | | KRT33B, | | KRT33B, | |
| KRT34 | | KRT34, | | KRT34, | |
| | | KRT35 | | KRT35, | |
| | | | | KRT36 | |
| KRT31, | KRT81, | KRT31, | KRT81, | KRT31, | KRT81, |
| KRT32, | KRT82 | KRT32, | KRT82 | KRT32, | KRT82 |
| KRT33A, | | KRT33A, | | KRT33A, | |
| KRT33B, | | KRT33B, | | KRT33B, | |
| KRT34 | | KRT34, | | KRT34, | |
| | | KRT35 | | KRT35, | |
| | | | | KRT36 | |
| KRT31, | KRT81, | KRT31, | KRT81, | KRT31, | KRT81, |
| KRT32, | KRT82, | KRT32, | KRT82, | KRT32, | KRT82, |
| KRT33A, | KRT83 | KRT33A, | KRT83 | KRT33A, | KRT83 |
| KRT33B, | | KRT33B, | | KRT33B, | |
| KRT34 | | KRT34, | | KRT34, | |
| | | KRT35 | | KRT35, | |
| | | | | KRT36 | |
| KRT31, | KRT81, | KRT31, | KRT81, | KRT31, | KRT81, |
| KRT32, | KRT82, | KRT32, | KRT82, | KRT32, | KRT82, |
| KRT33A, | KRT83, | KRT33A, | KRT83, | KRT33A, | KRT83, |
| KRT33B, | KRT84 | KRT33B, | KRT84 | KRT33B, | KRT84 |
| KRT34 | | KRT34, | | KRT34, | |
| | | KRT35 | | KRT35, | |
| | | | | KRT36 | |
| KRT31, | KRT81, | KRT31, | KRT81, | KRT31, | KRT81, |
| KRT32, | KRT82, | KRT32, | KRT82, | KRT32, | KRT82, |
| KRT33A, | KRT83, | KRT33A, | KRT83, | KRT33A, | KRT83, |
| KRT33B, | KRT84, | KRT33B, | KRT84, | KRT33B, | KRT84, |
| KRT34 | KRT85 | KRT34, | KRT85 | KRT34, | KRT85 |
| | | KRT35 | | KRT35, | |
| | | | | KRT36 | |
| KRT31, | KRT81, | KRT31, | KRT81, | KRT31, | KRT81, |
| KRT32, | KRT82, | KRT32, | KRT82, | KRT32, | KRT82, |
| KRT33A, | KRT83, | KRT33A, | KRT83, | KRT33A, | KRT83, |
| KRT33B, | KRT84, | KRT33B, | KRT84, | KRT33B, | KRT84, |
| KRT34 | KRT85, | KRT34, | KRT85, | KRT34, | KRT85, |
| | KRT86 | KRT35 | KRT86 | KRT35, | KRT86 |
| | | | | KRT36 | |

TABLE 2g

| Keratin filament | | Keratin filament | |
|---|---|---|---|
| network | | network | |
| Type 1 keratin | Type 2 keratin | Type 1 keratin | Type 2 keratin |
| subtype | subtype | subtype | subtype |
| KRT31, KRT32, | KRT81 | KRT31, KRT32, | KRT81 |
| KRT33A, KRT33B, | | KRT33A, KRT33B, | |
| KRT34, KRT35, | | KRT34, KRT35, | |
| KRT36, KRT37 | | KRT36, KRT37, | |
| | | KRT38 | |
| KRT31, KRT32, | KRT81, KRT82 | KRT31, KRT32, | KRT81, KRT82 |
| KRT33A, KRT33B, | | KRT33A, KRT33B, | |
| KRT34, KRT35, | | KRT34, KRT35, | |
| KRT36, KRT37 | | KRT36, KRT37, | |
| | | KRT38 | |
| KRT31, KRT32, | KRT81, KRT82, | KRT31, KRT32, | KRT81, KRT82, |
| KRT33A, KRT33B, | KRT83 | KRT33A, KRT33B, | KRT83 |
| KRT34, KRT35, | | KRT34, KRT35, | |
| KRT36, KRT37 | | KRT36, KRT37, | |
| | | KRT38 | |
| KRT31, KRT32, | KRT81, KRT82, | KRT31, KRT32, | KRT81, KRT82, |
| KRT33A, KRT33B, | KRT83, KRT84 | KRT33A, KRT33B, | KRT83, KRT84 |
| KRT34, KRT35, | | KRT34, KRT35, | |
| KRT36, KRT37 | | KRT36, KRT37, | |
| | | KRT38 | |
| KRT31, KRT32, | KRT81, KRT82, | KRT31, KRT32, | KRT81, KRT82, |
| KRT33A, KRT33B, | KRT83, KRT84, | KRT33A, KRT33B, | KRT83, KRT84, |
| KRT34, KRT35, | KRT85 | KRT34, KRT35, | KRT85 |
| KRT36, KRT37 | | KRT36, KRT37, | |
| | | KRT38 | |
| KRT31, KRT32, | KRT81, KRT82, | KRT31, KRT32, | KRT81, KRT82, |
| KRT33A, KRT33B, | KRT83, KRT84, | KRT33A, KRT33B, | KRT83, KRT84, |
| KRT34, KRT35, | KRT85, KRT86 | KRT34, KRT35, | KRT85, KRT86 |
| KRT36, KRT37 | | KRT36, KRT37, | |
| | | KRT38 | |

TABLE 2h

| Keratin filament | | Keratin filament | |
|---|---|---|---|
| network | | network | |
| Type 1 keratin | Type 2 keratin | Type 1 keratin | Type 2 keratin |
| subtype | subtype | subtype | subtype |
| KRT31, KRT32, | KRT81 | KRT31, KRT32, | KRT81 |
| KRT33A, KRT33B, | | KRT33A, KRT33B, | |
| KRT34, KRT35, | | KRT34, KRT35, | |
| KRT36, KRT37, | | KRT36, KRT37, | |
| KRT38, KRT39 | | KRT38, KRT39, | |
| | | KRT40T31 | |
| KRT31, KRT32, | KRT81, KRT82 | KRT31, KRT32, | KRT81, KRT82 |
| KRT33A, KRT33B, | | KRT33A, KRT33B, | |
| KRT34, KRT35, | | KRT34, KRT35, | |
| KRT36, KRT37, | | KRT36, KRT37, | |
| KRT38, KRT39 | | KRT38, KRT39 | |
| KRT31, KRT32, | KRT81, KRT82, | KRT31, KRT32, | KRT81, KRT82, |
| KRT33A, KRT33B, | KRT83 | KRT33A, KRT33B, | KRT83 |
| KRT34, KRT35, | | KRT34, KRT35, | |
| KRT36, KRT37, | | KRT36, KRT37, | |
| KRT38, KRT39 | | KRT38, KRT39 | |
| KRT31, KRT32, | KRT81, KRT82, | KRT31, KRT32, | KRT81, KRT82, |
| KRT33A, KRT33B, | KRT83, KRT84 | KRT33A, KRT33B, | KRT83, KRT84 |
| KRT34, KRT35, | | KRT34, KRT35, | |
| KRT36, KRT37, | | KRT36, KRT37, | |
| KRT38, KRT39 | | KRT38, KRT39 | |
| KRT31, KRT32, | KRT81, KRT82, | KRT31, KRT32, | KRT81, KRT82, |
| KRT33A, KRT33B, | KRT83, KRT84, | KRT33A, KRT33B, | KRT83, KRT84, |
| KRT34, KRT35, | KRT85 | KRT34, KRT35, | KRT85 |
| KRT36, KRT37, | | KRT36, KRT37, | |
| KRT38, KRT39 | | KRT38, KRT39 | |
| KRT31, KRT32, | KRT81, KRT82, | KRT31, KRT32, | KRT81, KRT82, |
| KRT33A, KRT33B, | KRT83, KRT84, | KRT33A, KRT33B, | KRT83, KRT84, |
| KRT34, KRT35, | KRT85, KRT86 | KRT34, KRT35, | KRT85, KRT86 |
| KRT36, KRT37, | | KRT36, KRT37, | |
| KRT38, KRT39 | | KRT38, KRT39 | |

The keratin filament network may comprise:
6 Type I human hair keratins selected from the group consisting of KRT31, KRT32, KRT33A, KRT33B, KRT34, and KRT35; and
6 Type II human hair keratins selected from the group consisting of KRT81, KRT82, KRT83, KRT84, KRT85 and KRT86.

The average filament (or nanofiber) diameter of the keratin filament network may be about 4 nm to about 15 nm. The average filament diameter of the keratin filament network may be about 4 nm to about 15 nm, about 4 nm to about 14 nm, about 4 nm to about 13 nm, about 4 nm to about 12 nm, about 4 nm to about 11 nm, about 4 nm to about 10 nm, about 4 nm to about 9 nm, about 4 nm to about 8 nm, about 4 nm to about 7 nm, about 4 nm to about 6 nm, about 4 nm to about 5 nm, about 5 nm to about 15 nm, about 6 nm to about 15 nm, about 7 nm to about 15 nm, about 8 nm to about 15 nm, about 9 nm to about 15 nm, about 10 nm to about 15 nm, about 11 nm to about 15 nm, about 12 nm to about 15 nm, about 13 nm to about 15 nm, about 14 nm to about 15 nm, or about 4 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, or any value or range therebetween.

The keratin filament network may be in a mesh form. The mesh may be an interlaced structure, or have a weblike pattern. The mesh may be made up of nanofibers that are of a narrow or wide distribution of diameters, wherein the average filament diameter is about 4 nm to about 15 nm.

The present invention also relates to a keratin filament network obtained by the method disclosed herein.

### EXAMPLES

Non-limiting examples of the invention and a comparative example will be further described in greater detail by reference to specific Examples, which should not be construed as in any way limiting the scope of the invention.

### Example 1: Extraction of Human Hair Keratins

Keratins were extracted from human hair following an extraction protocol as follows: delipidized hair was first incubated in a keratin-associated protein (KAP) extraction solution made up of 25 mM Tris-HCl buffer, pH 9.5 (Sigma), 8 M urea (Chem-Impex), 200 mM dithiothreitol (DTT) (GoldBiotechnology), and 25% ethanol at 50° C. for 72 hours. The KAP-free hair residues were washed thoroughly with deionized (DI) water and left to air-dry prior to the subsequent keratin extraction procedure. KAP-free hair was added to a pH 8.5 Tris-HCl buffer consisting of 5 M urea, 2.6 M thiourea (Sigma), and 200 mM DTT for 24 hours at 50° C. The extracted keratin mixture was then centrifuged at 13,000 rpm for 15 minutes and dialyzed against acidic buffers herein: citric acid, acetic acid, formic acid, lactic acid or other weak acids, via the step down dialysis approach, in a cellulose tubing of 10 kDa molecular weight cut-off.

### Example 2: Step Down Dialysis

The acidic buffer solution used for keratin self-assembly was prepared using citric acid (Sigma) or other weak acids such as acetic acid, formic acid and lactic acid, to produce buffers of pH 2.5-5.5. Taking citric acid as an example, a range of concentrations of 0.7-50 mM were prepared (e.g. 0.7 mM, 2.5 mM and 50 mM) to obtain buffers of pH of range 2.3-5.4 (e.g. 2.5, 2.9, 3.3, 4.5 and 5.5). For the acidic buffer solutions of pH 4.5 and pH 5.5, sodium hydroxide (NaOH) was used to adjust the pH of the 0.7 mM citric acid solution to achieve the desired pH values.

The extracted keratin solution was first dialyzed overnight against 8M urea and 1 mM DTT constituted in the acid buffer. Subsequently, the keratin solution was further dialyzed against dialysis buffers of lowered urea concentrations in a step-wise manner, from 4M to 2M, for 3-4 hours at each step. The keratin solution was left overnight in the final dialysis buffer (OM urea) for complete urea removal.

### Example 3: Incorporation of Self-Assembly Buffer Solution

Concentrations of the keratin solutions after step down dialysis were obtained using the Bradford assay (Biorad). Keratins were diluted to approximately 0.8-1 mg/ml with their corresponding dialysis buffers and filtered through a 0.22 um syringe filter before initiation of self-assembly by adding an equal volume of 5-40 mM KCl (e.g. 5 mM KCl or 40 mM KCl) dissolved in the corresponding buffer solution (hereon referred to as SA solution), for a few minutes up to a few hours. Fiber diameter was found to increase with 40 mM KCl compared to 5 mM KCl in the pH 3.3 group. Network connectivity improved with 40 mM KCl compared to 5 mM KCl in the pH 5.5 group.

After incubating in the SA solution for the desired duration, the keratin solution was added to a same volume of fixing solution containing 0.2% glutaraldehyde in the corresponding KCl concentration, for 3-5 minutes, and thereon ready to be applied to the intended surface to deposit the self-assembled networks. For characterization, this was done on glow discharged carbon coated grids and negatively stained with 2% uranyl acetate solution for TEM imaging.

### Example 4: Fixation and Sample Preparation for Transmission Electron Microscopy (TEM) Imaging

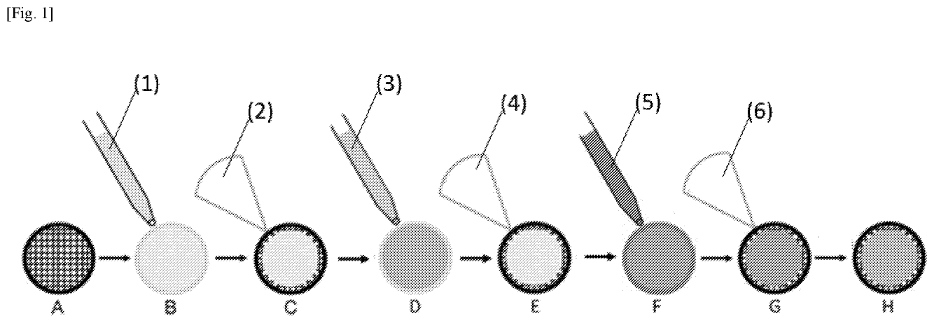

Referring to FIG. 1, FIG. 1 shows a schematic diagram of the staining procedures for TEM imaging of the keratin filament network. Type A-carbon coated grid (Ted Pella) (A) was glow-discharged for 60 seconds before use. Following from Example 3, after mixing the keratin solution with a same volume of fixing solution containing 0.2% glutaraldehyde and corresponding KCl concentration for 3-5 minutes. The final concentration of the keratin solution is 0.2-0.25 mg/mL while the final concentration of KCl in the keratin solution is half of its initial concentration.

5 μL of KIFP solution (1) (or SA-keratin solution) was added onto the grid and the solution was incubated for 30 seconds (Step B) followed by a 4 seconds blotting with filter paper (2) to remove excessive solution (Step C). The grid was then washed with 5 μL of deionized (DI) water (3) for 30 seconds (Step D) followed by 4 seconds blotting of excess solution using a filter paper (4) (Step E). 5 μL of 2% uranyl acetate solution (5) was used to negatively stain the KIFP solution for 30 seconds (Step F), followed by a 4 seconds blotting with filter paper (6) (Step G). The grid with the stained protein was left to dry in air for at least 15 minutes before storing in a desiccator overnight prior to TEM imaging (Step H).

### Example 5: Step By Step Procedure

### Step 1

Acidic buffers were prepared from a weak acid (such as citric acid, acetic acid, formic acid, or lactic acid). A range of concentrations were prepared to give different pH values (50 mM-pH 2.5; 2.5 mM-pH 2.9; 0.7 mM-pH 3.3; 0.7 mM adjusted with NaOH-pH 4.5/pH 5.5).

### Step 2

8 M Urea and 1 mM DTT were dissolved in the prepared acid buffer of Step 1 to form a dialysis buffer. The keratin solution was dialyzed in this dialysis buffer overnight.

### Step 3

4 M Urea and 1 mM DTT were dissolved in the prepared acid buffer. The keratin solution was dialyzed in this dialysis buffer for 3-4 hours.

### Step 4

2 M Urea and 1 mM DTT were dissolved in the prepared acid buffer. The keratin solution was dialyzed in this dialysis buffer for 3-4hours.

### Step 5

1 mM DTT was dissolved in the prepared acid buffer. The keratin solution was dialyzed in this dialysis buffer overnight.

Steps 1 to 5 describe the step-down dialysis procedure.

### Step 6

After Steps 1-5, the keratin solution was diluted to 0.8-1 mg/ml before initiation of the self-assembly process.

### Step 7

A self-assembly solution (SA solution) was prepared by dissolving 5-40 mM KCl in the acidic buffer mentioned in Step 1.

### Step 8

The diluted keratin from Step 6 was mixed with the SA solution (1:1) for the required duration (for example, about 1 minute to about 2 hours) for self-assembly.

Steps 6 to 8 describe the procedure to initiate self-assembly.

### Step 9

For TEM imaging, a fixing solution was prepared by dissolving 0.2% glutaraldehyde in the acidic buffer in Step 1.

### Step 10

The keratin solution with a same volume of fixing solution containing 0.2% glutaraldehyde and 2.5 mM KCl or 20 mM KCl were mixed for 3-5min.

### Step 11

The final concentration of the keratin solution after Steps 6-10 was 0.2 mg/ml to 0.25 mg/ml.

### Step 12

The final concentration of KCl in the keratin solution was half of its concentration stated in Step 7.

### Step 13

The self-assembled keratin fibers, after fixation in Step 10, were used for UA staining and TEM imaging.

### Example 6: Results of Hair Keratins After Undergoing Step Down Dialysis and Initiation of Self-Assembly in Self-Assembly (SA) Solution

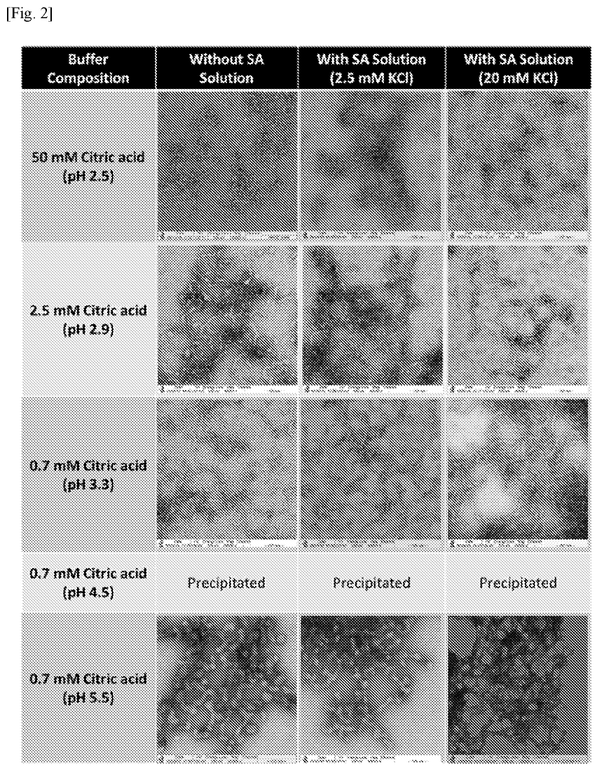

Referring to FIG. 2, FIG. 2 shows TEM images of hair keratins after undergoing step down dialysis and initiation of self-assembly in different compositions of potassium chloride, KCl (0 mM KCl, 2.5 mM KCl and 20 mM KCl) and acidic pH (2.5, 2.9, 3.3, 4.5 and 5.5) of the acidic buffer solution for an hour. The images in FIG. 2 show conformation of the hair keratins and that the human hair extracted keratins were able to self-assemble into filamentous structures, with filament diameters of 4-15 nm. Such self-assembled structures were observed when keratins were dialyzed in 0.7-50 mM citric acid buffer with 1 mM dithiothreitol (DTT), before introducing the acidic buffer solution. Upon additional of the acidic buffer solution, the filament smoothness and regularity were improved with increasing salt (potassium chloride, KCl) concentration. Bead-like structures were observed when keratins were dialysed at pH 5.5 due to isoelectric point (pI) precipitation (where pI of keratin is 4.5 to 5). Such grainy features showed improved connectivity upon the introduction of acidic buffer solution and with increasing concentrations of salt (KCl). As high concentration of salt (20 mM KCl) can disturb the charges on the protein molecules and trigger aggregation, keratins at pH close to the pI (e.g. pH 5.5) agglomorated when added to high salt content SA buffer solutions. However, the keratins at this condition still organized into network structures despite the agglomoration.

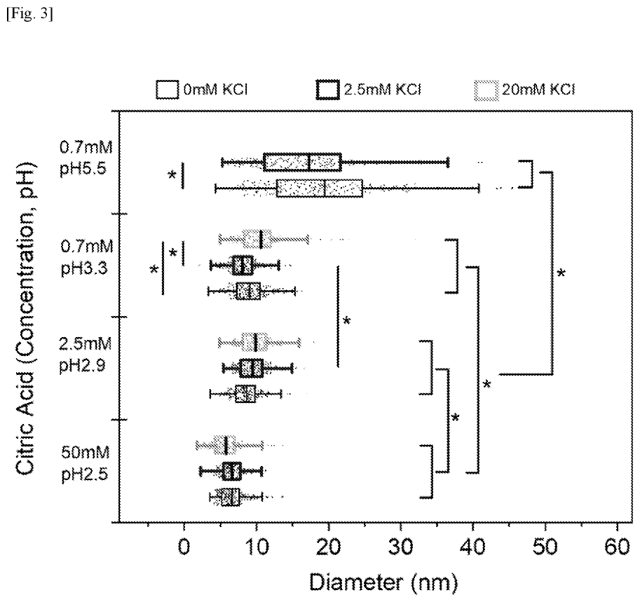

FIG. 3 shows box plots of SA-keratin nanofiber diameters across various self-assembly conditions in self-assembly (SA) acidic buffer solution. The minimum and maximum boundary lines of each box indicate the 25th and 75th percentile values, respectively. The lines within the boxes mark the mean values. Whiskers (above and below each box) indicate 1.5 interquartile range (IQR). *p<0.05, two-way ANOVA, Tukey's HSD post hoc test. Table 3 shows the tabulation of quantitative data of mean diameters±standard deviation (SD) (n=200) of SA-keratin nanofiber diameters across various self-assembly conditions in the SA acidic buffer solution.

TABLE 3
Quantitative data of mean diameters ± SD (n =
200) of SA-keratin nanofiber diameters across various
self-assembly conditions in the SA acidic buffer solution

| Buffer Composition | | | |
|---|---|---|---|
| Citric Acid | | | |
| Concentration and | | | |
| pH | 0 mM KCl | 2.5 mM KCl | 20 mM KCl |
| 0.7 mM Citric acid | 19.5 ± 8.7 nm | 17.3 ± 7.7 nm | Precipitated |
| pH 5.5 | | | |
| 0.7 mM Citric acid | 9.1 ± 2.5 nm | 8.0 ± 2.1 nm | 10.7 ± 3.6 nm |
| pH 3.3 | | | |
| 2.5 mM Citric acid | 8.6 ± 2.1 nm | 9.5 ± 2.1 nm | 9.8 ± 2.3 nm |
| pH 2.9 | | | |
| 50 mM Citric acid | 6.6 ± 1.9 nm | 6.6 ± 1.9 nm | 5.8 ± 2.2 nm |
| pH 2.5 | | | |

In acidic conditions (without the acidic buffer solution), nanofiber diameters were reduced proportionally with buffer pH, as evidenced in FIG. 3 and Table 3. The results in FIG. 3 and Table 3 show the change in average fiber diameter from 9.1±2.5 nm to 6.6±1.9 nm (p<0.01) when the pH was reduced from 3.3 to 2.5. In contrast, increasing the salt concentration in the SA solution yielded significantly thicker fibers in 0.7 mM citric acid condition (pH 3.3). This was evidenced by the change of fiber diameter from 9.1±2.5 nm to 10.7±3.6 nm (p<0.01) when salt concentration was increased from 0 mM to 20 mM. Employing the method of the invention, the assembled and elongated hair keratin nanofibers were found to be comparable to self-assembled purified epidermal keratins and specific recombinant hair keratin subtypes. On the contrary, short fibers (<50 nm) with beaded ends and average diameter of 19.5 nm were observed when keratins were dialyzed at pH 5.5, possibly due to isoelectric point (keratin pI: 4.5-5.5) precipitation. Improved connectivity between the beads and bead elongation were observed under TEM upon the introduction of 2.5 mM KCl, resulting in fiber networks of smaller fiber diameters. High ionic charge, however, have been demonstrated to perturb the charges of the protein molecules and trigger aggregation. This effect was specifically apparent when the environment pH approached the protein pI values, during which the protein becomes more susceptible to precipitation due to the reduction of stabilizing electrostatic repulsion forces. Indeed, agglomeration was observed upon addition of keratin solutions (at pH 5.5) into high salt content SA solutions, albeit network structures were maintained.

### Example 7: pH Dependent Analysis of Dialysis Acidic Buffer

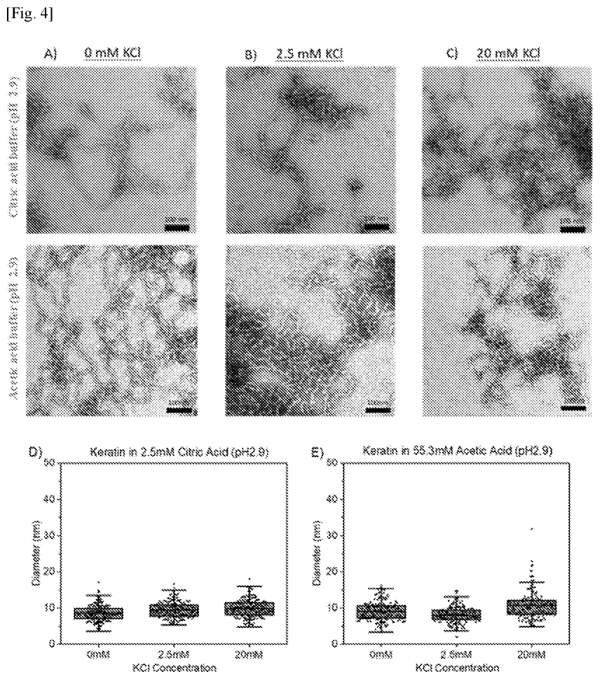

Referring to FIGS. 4A-4E, FIGS. 4A-4C show representative TEM images of self-assembled keratin fibers prepared in 2.5 mM citric acid and 55.3 mM acetic acid buffer, both at pH 2.9, while FIGS. 4D and 4E shows the box plots of SA-keratin nanofiber diameters prepared in (D) 2.5 mM citric acid and (E) 55.3 mM acetic acid buffer. The minimum and maximum boundary lines of each box indicate the 25th and 75th percentile values, respectively. The lines within the boxes mark the mean values. Whiskers (above and below each box) indicate 1.5 interquartile range (IQR).

To verify that the self-assembly event was mainly pH dependent, a strong acid (1.25 mM hydrochloric acid (HCl)) and a weak acid (55.3 mM acetic acid), were used in replacement of citric acid to adjust the dialysis buffer to pH 2.9. Among the three acids tested, HCl showed the least buffering ability against high molarity urea, resulting in the precipitation of keratins during dialysis and the absence of any self-assembled fibers. Hence, weak acids are preferred (e.g. citric acid, acetic acid, formic acid, ascorbic acid, benzoic acid, propionic acid, sorbic acid, maleic acid, gallic acid and/or lactic acid).

Nonetheless, SA-keratin formed in 55.3 mM acetic acid, pH 2.9, condition showed comparable fiber diameters, which range from 8.1 to 8.9 nm to the SA-keratin formed in 2.5 mM citric acid.

TABLE 4
Quantitative data of mean diameters ± SD (n =
200) of SA-keratin nanofiber diameters across various
self-assembly conditions in the SA acidic buffer solution

| Buffer Composition | | | |
|---|---|---|---|
| Acid | | | |
| Conc. & pH | 0 mM KCl | 2.5 mM KCl | 20 mM KCl |
| 2.5 mM Citric acid | 8.6 ± 2.1 nm | 9.5 ± 2.1 nm | 9.8 ± 2.3 nm |
| pH 2.9 | | | |
| 55.3 mM Acetic acid | 8.9 ± 2.6 | 8.1 ± 2.7 | 8.5 ± 2.7 |
| pH 2.9 | | | |

### Example 8: Characterization of SA-Keratin Prepared in 2.5 mM Citric Acid at pH 2.9

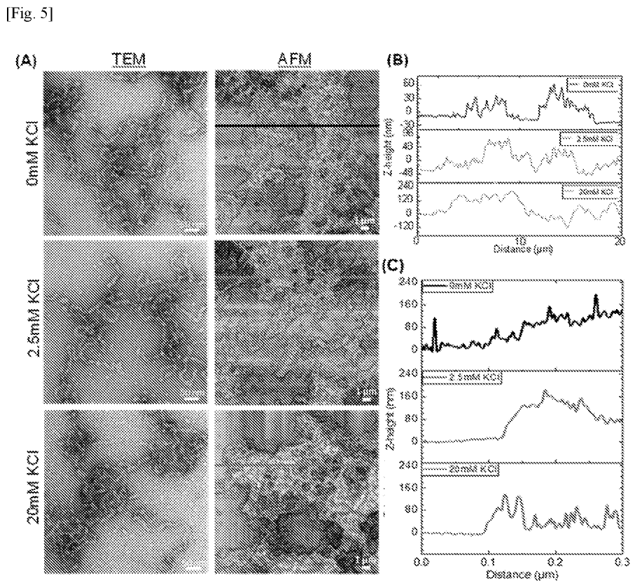

FIG. 5A shows representative TEM and atomic force microscopy (AFM) images of SA-keratins prepared in 2.5 mM citric acid buffer (pH 2.9) following a 1 hour self-assembly process. FIGS. 5B and 5C shows the Z-height profile obtained from AFM line scans and surface profilometer scans respectively.

Among the acidic buffer conditions tested, the SA-keratin prepared in 2.5 mM citric acid (pH 2.9) showed the greatest homogeneity and consistency in term of fiber morphology and diameter. Hence, this condition was selected for the subsequent coating deposition and in vitro studies. Consistent with TEM observations shown in FIG. 5A, the SA-keratin nanofibers formed at this condition were observed as filamentous networks as well using atomic force microscopy (AFM). In FIG. 5B, the Z-heights of the SA-keratin coatings were obtained using the AFM mapping analysis. Further, to ensure good observation and quantification of fiber morphology and diameter, the images presented in FIG. 5 were intentionally captured at areas that were more sparsely coated with fibers. This led to a heterogeneous distribution of the coatings on silicon wafer, with measured thickness of the entire layer ranging between 30-200 nm (FIG. 5B). The thickness of the coating was further measured using a surface profilometer as observed in FIG. 5C, which yielded comparable values to the AFM result.

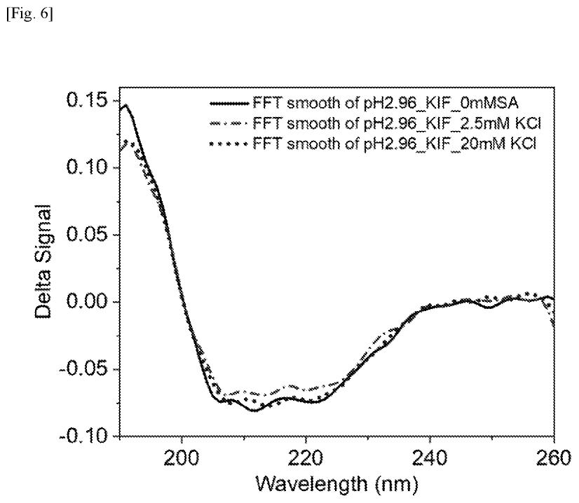

Further analysis with circular dichroism (CD) as shown in FIG. 6 revealed that the SA-keratins were mainly composed of alpha-helix domains.

### Example 9: Analysis of Self-Assembly Initiation

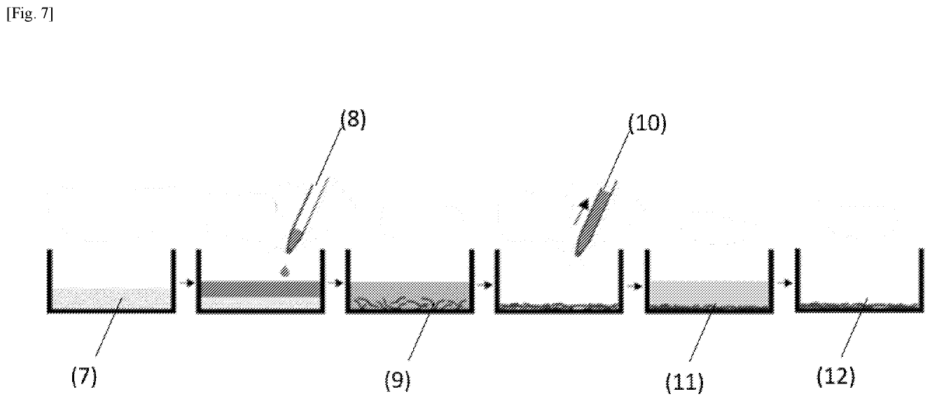

FIG. 7 shows the procedure of SA-keratin coating onto tissue culture plates. An acidic buffer solution (7) was provided. Keratin solution (8) was added to the acidic buffer solution and left at room temperature for 1 hour. Self-assembly of the keratin filament network occurred (9). After an hour, the solution was removed (10) and the keratin filament network was rinsed (11) and allowed to air dry (12).

In order to verify if the procedure described in FIG. 7 where a homogenous keratin thin film can be formed in 24-well plates after one hour of self-assembly initiation, primary anti-hair cortex cytokeratin antibodies (AE13, dilution 1:250) were used to perform immunoperoxidase staining.

Different concentrations of keratin intermediate filament proteins (KIFPs) and salt in the SA buffer were prepared to investigate the self-assembly initiation process. The different sample conditions investigated are tabulated in Table 5.

TABLE 5
Different sample conditions to investigate
the self-assembly initiation process

| Sample No. | Sample Condition |
|---|---|
| 1 | Negative Control (with human hair cytokeratin |
| | protein marker, AE13 ab) |
| 2 | 1 mg/mL KIFPs + 2.5 mM KCl SA Buffer (1 hour) |
| 3 | 1 mg/mL KIFPs + 20 mM KCl SA Buffer (1 hour) |
| 4 | 2 mg/mL KIFPs + 2.5 mM KCl SA Buffer (1 hour) |
| 5 | 2 mg/mL KIFPs + 20 mM KCl SA Buffer (1 hour) |

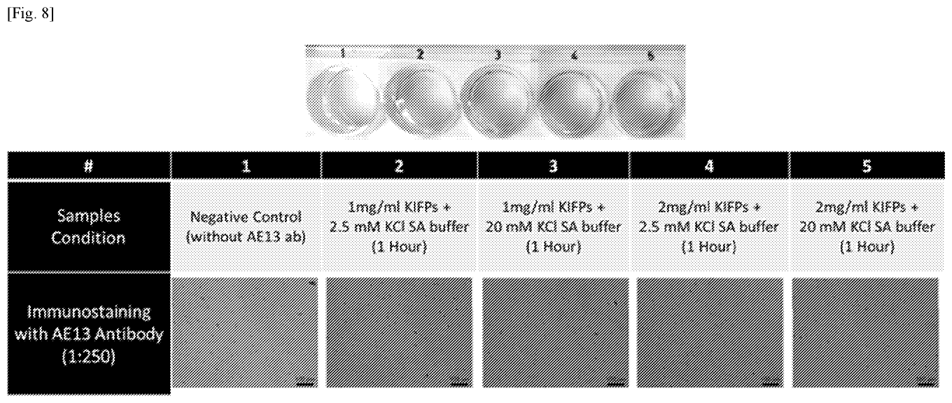

FIG. 8 shows the immunoperoxidase staining and formation of thin films after mixing hair keratins with SA acidic buffer solutions for 1 hour. It shows the results of the immunoperoxidase staining where positive staining can be observed in keratin samples 2-4 (where it consists of keratin intermediate filament proteins of concentration 1 mg/ml and 2 mg/ml) added to both SA buffers consisting 2.5 mM and 20 mM KCl, while the negative control (sample 1) remained unstained. This indicates the deposition of a stable and homogenous keratin thin film on the well plate surfaces.

### Example 10: Study of Efficiency and Stability of Coating Deposition at Different pH

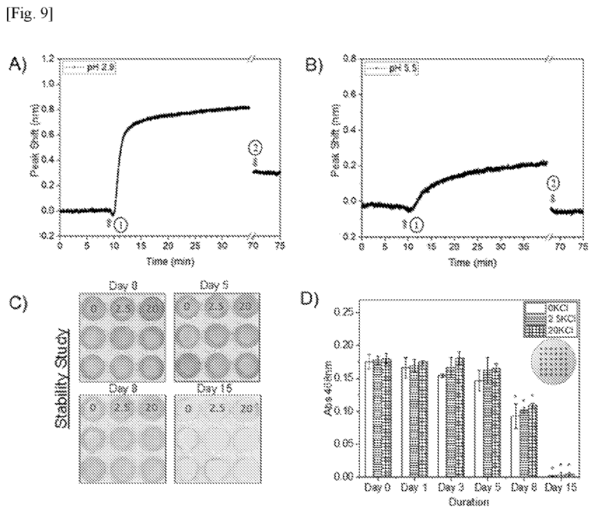

The adsorption kinetics of SA-keratin coatings as described in FIG. 7 were monitored via Indirect Nanoplasmonic Sensing (INPS). FIGS. 9A and 9B show the INPS analysis of coating deposition using SA-keratin solution at pH of 2.9 and 5.5. FIGS. 9A and 9B also indicates two time points where (1) indicates keratin was injected and flow was paused; and (2) indicates where after 8M urea rinse was performed.

As shown in FIG. 9A, SA-keratin fibers at pH 2.9 were observed to achieve pseudo-saturation 22 minutes after the flow was paused (at time point 1). The deposition of SA-keratin coating was noted by the shift of the peak signal, which is proportional to the change in refractive index contributed by the adsorbed proteins. Higher initial absorption rate was observed in SA-keratin formed at pH 2.9 in contrast to the keratin at pH 5.5, evident from the steeper gradient (m at pH 2.9=0.499 vs. m at pH 5.5=0.036) captured from the 10th min onward. This could be attributed to the stronger electrostatic interaction between the positively charged SA-keratin (pH 2.9) and the sensor chip surface, compared to the neutral keratin at pI condition at pH 5.5. Moreover, an increment of 0.8 nm peak shifted was observed at the saturated point and further declined to 0.3 nm following the rinsing steps, which removed loosely bound keratin molecules. Close to 40% of the coating remained on the sensor chip even after extensive flushing, suggesting strong adhesion of keratins on the sensor chip. In contrast, under the same INPS experimental conditions as shown in FIG. 9B, keratin at pH 5.5 showed a smaller peak shift at the saturation point (0.3 nm) and minimal attachment (11%) on the sensor chip after urea rinse (at time point 2). This could be attributed to the charge effect and bead-like morphology that led to weaker adhesion of keratin molecules on the sensor chip surface. These observations further support the conclusion that 2.5 mM citric acid at pH 2.9 produced the most optimal self-assembled keratin fiber networks.

Moreover, coating stability and biocompatibility are the basic evaluation parameters required to understand their potential for biomedical applications. As such, the stability of SA-keratin coatings was analyzed in vitro in complete DMEM cell culture media at physiological temperature (37° C.). FIG. 9C shows the successful formation of this uniform and good coverage SA-keratin coating could be observed by immunoperoxidase staining of broad spectrum human hair keratins, brown colour indicates a positive stain by human hair cytokeratin protein marker (AE 13). Further, the positive stainings were relatively constant up to 5 days of incubation, suggesting stability of the coating over this timeframe. FIG. 9D shows that significant reduction in staining intensity was noted at day 8, when close to 50% reduction of the immunoperoxidase staining was registered. Representative digital images revealed that coatings deposited at the center of the culture wells were effectively lost at day 15, confirmed by the significant reduction in immunoperoxidase staining based on absorbance mapping as observed in FIGS. 9C and 9D.

### Example 11: Biocompatibility Study of SA-Keratin Coatings

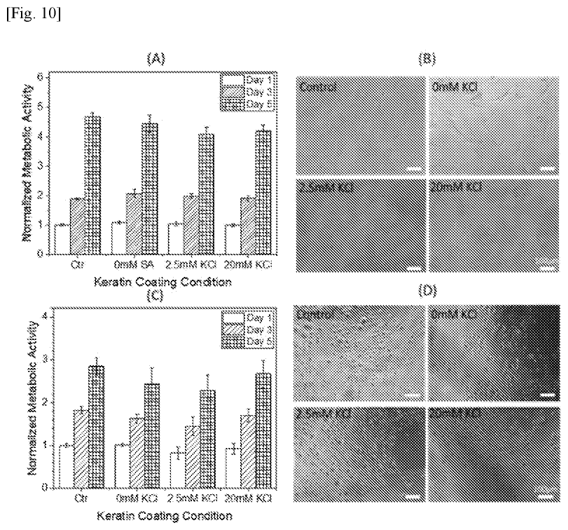

In vitro biocompatibility of the SA-keratin coatings was tested with human dermal fibroblasts (HDF) and human epidermal keratinocytes (HEK), over the course of 5 days. FIG. 10 shows the biocompatibility results of SA-keratin coating at pH 2.9 for (A and B) HDF and (C and D) HEK cells (n=3). The levels of metabolic activity of the cells grown on the coatings were comparable to controls cultured on untreated surfaces, as shown in FIGS. 10A and 10C. Metabolic activity of (A) HDF and (C) HEK as observed in FIGS. 10A and 10C are studied over the course of 5 days. Brightfield images showing the respective cell morphologies of HDF and HEK grown on SA-keratin coatings on day 5 are observed in FIGS. 10B and 10D. Keratin-derived biomaterials have been reported to facilitate wound healing and in more recent studies have been demonstrated to possess antioxidant properties. Herein, the expression of extracellular matrix (ECM) proteins, fibronectin and focal adhesion proteins, vinculin of HDF and HEK were investigated.

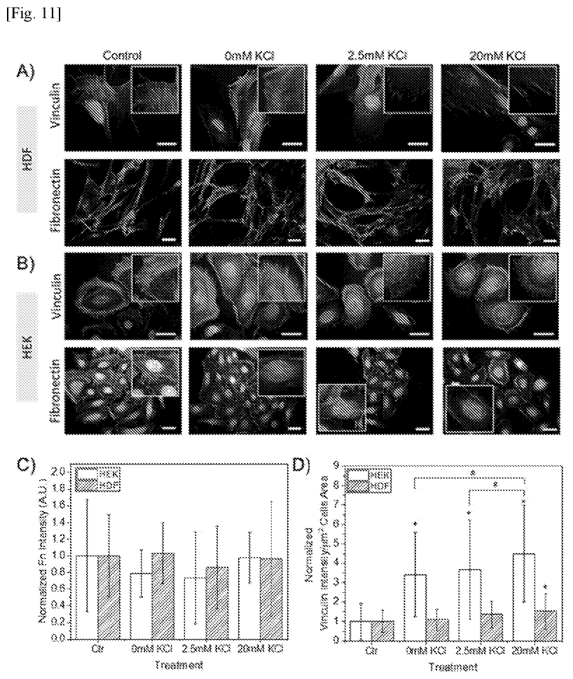

FIGS. 11A and 11B show the representative immunofluorescent images of (A) HDF and (B) HEK grown on SA-keratin coating (pH 2.9). FIGS. 11C and 11D shows the staining intensities of (C) fibronectin and (D) vinculin expressed by HDF and HEK. The staining intensities were quantified using ImageJ, with scale bar of 25 μm.

Fibronectin was found to be expressed in both HDF and HEK. With reference to FIGS. 11A and 11B, ECM protein fibronectin and focal adhesion protein vinculin were visualized with immunofluorescence. The actin network and nuclei were stained, respectively (light areas/light coloured nuclei). Although the fibronectin expression of the HDF and HEK cells did not show any significant differences as observed in FIG. 11C, an increase in the vinculin intensity (FIG. 11D) was noted between the control and the coating groups. This indicated the cell adhesion of both HDF and HEK were enhanced on the SA-keratin coatings in comparison to the control. These preliminary results indicate the casted coating have great potential to be applied in biomedical or cosmetic related applications.

### Example 12: Characterization of Keratin Using Gel Electrophoresis and Mass Spectrometry

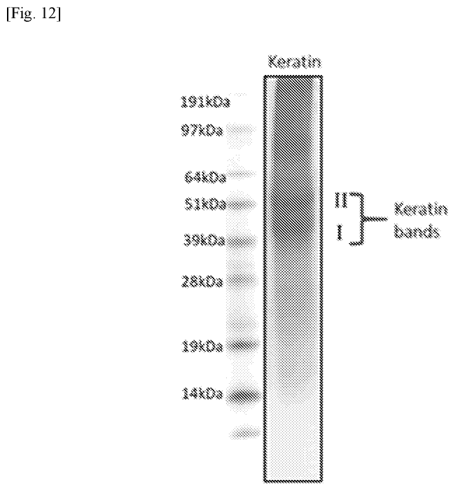

The extracted keratin solution was subjected to gel electrophoresis (SDS-PAGE) and the protein bands were excised, digested and processed for MALDI-ToF (matrix assisted laser desorption ionization-time of flight) mass spectrometry for protein identification. FIG. 12 shows the SDS PAGE and Coomassie blue staining profile of human hair keratins. It indicates the presence of type I and II keratins by two distinct bands on the SDS PAGE gel.

TABLE 6
Protein identification analysis of
human hair keratin using MALDI-ToF

| | | | Protein | Pro- |
|---|---|---|---|---|
| Acces- | Gene | | mass | tein |
| sion | Symbol | Protein Name | (kDa) | score |
| Q15323 | KRT31 | Keratin, type I cuticular Ha1 | 48.632 | 576 |
| Q14532 | KRT32 | Keratin, type I cuticular Ha2 | 51.79 | 95 |
| O76009 | KRT33A | Keratin, type I cuticular Ha3-I | 47.17 | 340 |
| Q14525 | KRT33B | Keratin, type I cuticular Ha3-II | 47.33 | 513 |
| O76011 | KRT34 | Keratin, type I cuticular Ha4 | 50.82 | 145 |
| Q92764 | KRT35 | Keratin, type I cuticular Ha5 | 51.64 | 85 |
| Q14533 | KRT81 | Keratin, type II cuticular Hb1 | 56.83 | 420 |
| Q9NSB4 | KRT82 | Keratin, type II cuticular Hb2 | 57.98 | 40 |
| P78385 | KRT83 | Keratin, type II cuticular Hb3 | 55.92 | 424 |
| Q9NSB2 | KRT84 | Keratin, type II cuticular Hb4 | 65.94 | 43 |
| P78386 | KRT85 | Keratin, type II cuticular Hb5 | 57.31 | 206 |
| O43790 | KRT86 | Keratin, type II cuticular Hb6 | 55.21 | 433 |

SwissProt-Human data base was used for data search. Among the eleven type I human hair keratins, KRT31, KRT32, KRT33A, KRT33B, KRT34, and KRT35 were detected in the extracted keratin samples (Table 6). On the other hand, all six type II keratins (KRT81, KRT82, KRT83, KRT84, KRT85 and KRT86) were detected. These identified keratin subtypes were therefore involved in the self-assembly process to form the filamentous network.

### INDUSTRIAL APPLICABILITY

The disclosed method allows the forming of a keratin filament network. Advantageously, the disclosed method is simple, less laborious, allows for high throughput and ease of up-scaling and can be utilized using solubilized human hair keratins which can be extracted from any keratin source. Further the invention generally relates to keratin filament networks. The present invention also relates to methods for making these keratin filament networks. The keratin filament network may be useful for medical, personal care, cosmetics and research applications.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of forming a keratin filament network, comprising:
(i) dialysing a solubilized keratin solution in a dialysis buffer solution at a pH of about 2.5 to about 5.5 to obtain purified keratin;
(ii) mixing the purified keratin with a salt in acidic buffer solution; and
(iii) drying the solution of step (ii) to form the keratin filament network.

2. The method of claim 1, wherein the method comprises self-assembly of the keratin filament network or wherein the solubilized keratin solution is substantially free of keratin-associated protein (KAP).

3. The method of claim 1, wherein the dialysis buffer solution comprises citric acid, acetic acid, formic acid, ascorbic acid, benzoic acid, propionic acid, sorbic acid, maleic acid, gallic acid and/or lactic acid; or wherein the dialysis buffer solution further comprises a denaturing agent or a reducing agent.

4. (canceled)

5. The method of claim 3, wherein the denaturing agent is urea or guanidine HCl.

6. The method of claim 3, wherein the starting concentration of the denaturing agent in the dialysis buffer solution is in the range of about 6M to about 10M.

7. The method of claim 1, wherein step (i) is performed for at least 2 hours; or wherein step (i) is repeated more than once.

8. (canceled)

9. The method of claim 6, wherein the concentration of denaturing agent in each repeated step (i) is lower than the concentration of denaturing agent in the preceding step (i); or wherein the concentration of denaturing agent in each repeated step (i) is about half the concentration of denaturing agent in the preceding step (i).

10. (canceled)

11. (canceled)

12. The method of claim 3, wherein the concentration of reducing agent in the dialysis buffer solution is about 0.5 mM to about 10 mM; or wherein the reducing agent is selected from the group consisting of dithiothreitol (DTT), beta-mercaptoethanol, tris(2-carboxyethyl)phosphine), and dithioerythritol (DTE).

13. (canceled)

14. The method of claim 1, wherein the acidic buffer solution comprises citric acid, acetic acid, formic acid, ascorbic acid, benzoic acid, propionic acid, sorbic acid, maleic acid, gallic acid and/or lactic acid.

15. The method of claim 1, wherein the concentration of the salt in the acidic buffer solution is about 1 mM to about 40 mM; or wherein the salt is selected from the group consisting of carbonate salts, sulfate salts, thiosulfate salts, phosphate salts, fluoride salts and chloride salts; or wherein the salt is selected from the group consisting of lithium chloride, sodium chloride, potassium chloride, calcium chloride, magnesium chloride, magnesium sulfate, ammonium sulfate, potassium carbonate, and calcium sulfate.

16. (canceled)

17. (canceled)

18. The method of claim 1, wherein
step (ii) comprises diluting the purified keratin prior to mixing; or
wherein step (ii) comprises mixing a substantially equal volume of diluted purified keratin with a substantially equal volume of solution of salt in an acidic buffer solution; or
wherein step (ii) is performed for a duration of about 1 minute to about 120 minutes.

19. The method of claim 18, wherein the purified keratin is diluted to about 0.5 mg/ml to about 100 mg/ml in the dialysis buffer solution.

20. (canceled)

21. (canceled)

22. The method of claim 1, wherein the method further comprises step (ia) extracting keratin from a keratin protein source with an extraction solution to form a solubilized keratin solution, wherein step (ia) occurs before step (i).

23. The method of claim 22, wherein the keratin protein source is selected from the group consisting of hair, wool, fur, horns, hooves, beaks, feathers, and scales.

24. (canceled)

25. A keratin filament network comprising at least 2 Type I human hair keratins and at least 2 Type II human hair keratins.

26. The keratin filament network of claim 25, comprising at least 4 Type I human hair keratins and at least 4 Type II human hair keratins.

27. The keratin filament network of claim 25, wherein the Type I human hair keratin is selected from the group consisting of KRT31, KRT32, KRT33A, KRT33B, KRT34, KRT35, KRT36, KRT37, KRT38, KRT39 and KRT40; or wherein the Type II human hair keratin is selected from the group consisting of KRT81, KRT82, KRT83, KRT84, KRT85 and KRT86.

28. (canceled)

29. The keratin filament network of claim 25 comprising:
6 Type I human hair keratins selected from the group consisting of KRT31, KRT32, KRT33A, KRT33B, KRT34, and KRT35; and
6 Type II human hair keratins selected from the group consisting of KRT81, KRT82, KRT83, KRT84, KRT85 and KRT86.

30. The keratin filament network of claim 25, wherein the average filament diameter is about 4 nm to about 15 nm; or wherein the network is a mesh.

31. (canceled)

32. A keratin filament network obtained by the method of claim 1.
